(19) United States
(12) Patent Application Publication
(10) Pub. No.: US 2004/0091477 A1
(43) Pub. Date: May 13, 2004

(54) Immunosuppresive compositions

(76) Inventors: David Haines, McLeans, VA (US); Arpad Tosaki, Debrecen (HU); Fadia F. Mahmound, Salawa Kuwait (KW)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/275,996

(22) PCT Filed: May 8, 2001
(86) PCT No.: PCT/US01/14718

(51) Int. Cl. A61K 39/395; A61K 38/13; A61K 31/4745; A61K 35/78

(52) U.S. Cl. 424/144.1; 424/752; 514/11; 514/291

(57) ABSTRACT

The invention features a composition containing an immunophilin-binding compound and a ginkgolide compound, methods of inducing immunosuppression, and methods of screening for immunosuppressive ginkgolide compounds. The ginkgolide compound is selected from the group consisting of a phosphodiesterase inhibitor, a platelet activating factor (PAFR) antagonist, and a free radical scavenger.

### BACKGROUND

The invention relates to immunosuppression.

Immunosuppressants such as cyclosporin A (CsA) and FK-506 have been used clinically to inhibit rejection of transplanted organs and to treat autoimmune diseases. In many cases, transplant recipients and individuals suffering from autoimmune diseases require long term immunosuppression. High toxicity profiles of immunosuppressants such as CsA and FK506 limit their clinical benefit. Side effects include nephrotoxicity, hypertension, increased risk of diabetes, and neurological toxicity.

### SUMMARY OF THE INVENTION

The invention features immunosuppressive compositions with reduced adverse side effects. The immunosuppressive composition contains an immunophilin-binding agent such as a calcineurin inhibitor and a ginkgolide. The gingkolide composition preferably contains an antioxidant or free radical scavenger. More preferably, the gingolide contains platelet activating factor receptor (PAFR) antagonist activity. Most preferably, the composition contains PAFR antagonist activity and at least two antioxidants. The composition contains a cyclic AMP phosphodiesterase inhibitor. For example, the gingkolide composition has the following activities: cAMP phophosdiesterase inhibitory activity, PAFR receptor antagonist activity, and antioxidant or free radical scavenging activity.

A free radical scavenger is a composition which reduces the level of free radicals. For example, a free radical scavenger reduces the level of oxygen radical which contribute to oxidative stress. The level of free radicals is reduced by direct scavenging of free radicals or by inducing the production of a composition with a scavenging effect on free radicals. A phosphodiesterase inhibitor is a composition, which inhibits or selectively reduces the activity of a phosphodiesterase enzyme. For example, the compound inhibits the enzymatic activity of a cAMP-specific phosphodiesterase. The compound inhibits type III phosphodiesterases or type IV phospodiesterases. A PAFR antagonist is a compound which inhibits binding of PAF (1-O-alkyl-2-acetyl-sn-glycerol-3-phosphorylcholine) to its receptor. PAF is a potent inflammatory phospholipid mediator, and inhibition of PAF binding to PAFR, e.g., by a PAFR antagonist, reduces inflammation.

Immunophilin-binding compositions include rapamycin or an analog thereof and calcineurin inhibitors such as FK506 and Cyclosporin A (CsA) or an analog thereof.

The invention also includes a method of inducing immunosuppression (e.g., inhibiting activation of an immune cell such as a T cell or a B cell) in a mammal. The method is carried out by coadministering of an immunophilin-binding compound such as a calcineurin inhibitor and a gingkolide in such amounts so as to provide a synergistic immunosuppressive effect. Preferably, the gingkolide is administered at a dose that preferentially inhibits T-cell activation. The immunophilin-binding compound (e.g., a calcineurin inhibitor) and the gingkolide are administered simultaneously or consecutively. For example, the gingkolide is first administered followed by the immunophilin-binding compound. Alternatively, the immunophilin-binding compound is administered first and then the gingkolide. Such a coadministration regimen is useful to inhibit rejection of an allografted tissue in a mammal. For example, a calcineurin inhibitor and a gingkolide are administered prior to or after transplantation of an allogeneic tissue.

A method of inhibiting activation of an immune cell is carried out by contacting an immune cell with an immunophilin-binding compound and a gingkolide compound. The ginkgolide compound is selected from the group consisting of a phosphodiesterase inhibitor, a platelet activating factor (PAFR) antagonist, and a free radical scavenger. Therapeutic methods include a method of inhibiting rejection of an allograft in a mammal; a method of inhibiting cardiac hypertrophy in a mammal; a method of reducing a symptom, e.g., chronic inflammation, of an autoimmune disease; and a method of reducing a symptom of asthma. A method of suppressing an immune response in a mamal is carried out by administering to the mammal an effective amount for inducing a synergistic immunosuppression of an immunophilin-binding compound and a gingkolide compound. The compounds are administerd simultaneously or sequentially, by the same or different routes.

Also within the invention are methods of screening for therapeutic compounds. A method of identifying an immunosuppressive agent is carried out by contacting an immune cell population with candidate ginkgolide compound and measuring T cell activation. A decrease in T cell activation in the presence of the compound compared to the level in the absence of the compound indicates that the compound is an immunosuppressive agent. For example, T cell activation is measured by detecting cell surface CD25 expression, or production of a cytokine such as IL-2. To identify a syngergistic combination of immunosuppressive compounds, the following steps are carried out: (a) contacting an immune cell with an immunophilin-binding compound; (b) contacting an immune cell with a candidate ginkgolide compound; (c) contacting an immune cell with both an immunophilin-binding compound and a candidate ginkgolide compound; and (d) measuring T cell activation. A greater than additive decrease in T cell activation detected in the cell culture in step (c) compared to that detected in the cell cultures of (a) and (b) indicates that the ginkgolide and immunophilin-binding compound tested are synergistically immunosuppressive.

Other features and advantages of the invention will be apparent from the following detailed description and from the claims.

### BRIEF DESCRIPTION OF THE DRAWINGS

FIG. 1 is a bar graph showing in vitro cytokine expression responses by PBMC from asthmatic subjects. Expression of interleukins 4 and 5 by activated, or non-activated human PBMC, to CsA and BN52021 were measured. Cells were cultured with media (solid columns), 20 nM PMA+1.0 μM ionomycin (PMA/I) (dark hatched columns), 1.0 μM CsA+PMA/I (grey columns), or 1 μM BN52021+PMA/I (light hatched columns). Concentrations of IL-4 and IL-5 (as a measure of immune activation) were determined in culture supernatants by antibody capture ELISA and expressed in pg/ml. Activated cells responded to CsA treatment by suppression of both cytokines, with a trend toward suppression resulting from treatment with BN52021.

FIG. 2 is a bar graph showing in vitro expression of lymphocyte activation markers in PBMC from asthma patients. Expression of lymphocyte surface activation markers by activated or nonactivated human PBMC was measured. Cells were stimulated with media (solid columns), 20 nM PMA+1.0 μM ionomycin (PMA/I) (dark hatched columns), 1.0 μM CsA+PMA/I (grey columns), or 1 μM BN52021+PMA/I (light hatched columns). After 24 hours flow cytometric analysis was conducted for expression of CD25, CD45RA, CD54 and HLA-DR. Results are representative of 3 independent assays and are expressed as the percentage of lymphocytes in each sample expressing a particular surface antigen.

FIG. 3A is a bar graph showing the incidence of total (reversible and irreversible) reperfusion-induced ventricular fibrillation (VF) (open columns) and incidence of irreversible VF (solid columns) relative to dose of FK506.

FIG. 3B is a bar graph showing the incidence of ventricular tachycardia (VT). Incidence of arrythmias indicates percentage of 12 hearts showing VF and VT during reperfusion. N=12 in each group. P<0.05 compared with drug-free controls.

FIG. 4A is a bar graph showing the incidence of total (reversible and irreversible) reperfusion-induced ventricular fibrillation (VF) (open columns) and incidence of irreversible VF (solid columns) relative to dose of FK506 and Egb761.

FIG. 4B is a bar graph showing the incidence of ventricular tachycardia (VT). Incidence of arrythmias indicates percentage of 12 hearts showing VF and VT during reperfusion. N=12 in each group. P<0.05 compared with drug-free controls.

### DETAILED DESCRIPTION

The compositions described herein are useful to prevent rejection of transplanted organs, prevent cardiac hypertrophy, and improve the clinical prognosis for patients suffering from ischemic heart disease The compositions are also useful to treat autoimmune diseases. The combined action of a macrolide immunosuppressant such as FK506 with one or more components of a Ginkgo biloba extract allows a lower dose of the macrolide immunosuppressant than that required to achieve immunsuppression in the absence of a gingkolide.

### Immunosuppressive Agents

IL-2 is an important messenger in the signal transduction pathway that leads to the stimulation and proliferation of T-cells. Once a T-cell is activated, a series of signals in the cytoplasm and nucleus leads to the production of IL-2 and IL-2 receptors. One of the steps in the signal transduction cascade is the activation of the transcription factor nuclear factor of activated T-cells (NFAT) by calcineurin. The immunosuppressive agents described herein inhibit T cell activation.

Calcineurin inhibitors include FK506 (Tacrolimus), L-732,531 (a semi-synthetic analog of FK506), FK 520, L-683, 590 (an analog of FK506), L-685,818 (a C18-hydroxy, C21-ethyl derivative of FK506), L-732,531 (an analog of FK506 containing a hydroxyethylindole substituent), 9-deoxo-31-O-demethyl FK506, 31 -O-demethyl FK506, FK1012, Ascomycin (a class of anti-inflammatory macrolactams), A-119435 (a less nephrotoxic analog of ascomycin with 10× higher therapeutic index), SDZ ASM 981 (an ascomycin derivative), indolyl-ASC,

Ascomycin with a 2-carbon tether, Ascomycin with a 2-carbon 9 tether and linked oxygen-bearing substituents; “tether”=C32-O-arylethyl ether), C24-deoxyascomycin, ABT-281, AKAP79 (an analoge which inhibits calcineurin through a site distinct from immunophilin-binding region), tyrphostins A8 (also designated AG10), tyrphostins A23 (also designated AG18), tyrphostins A48 (also designated AG112)

### Cyclosporin A and Analogs

Cyclosporin A is produced as a metabolite by the fungus species Tolypocladium inflatum Gams. Chemically, cyclosporin A is designated as [R-[R,R-(E)]]-cyclic-(L-alanyl-D-alanyl-N-methyl-L-leucyl-N-methyl-L -leucyl-1N-methyl-L-valyl-3-hydroxyN,4-dimethyl-L-2-amino-6-octenoyl-L-a-amino-butyryl-N-methylglycyl-N-methyl-L-leucyl-L-valyl-N-methyl-L-leucyl). Cyclosporin A is commercially available in oral dosage form or intravenous dosage form Cyclosporin forms a complex with cyclophilin in the cytoplasm, which then binds to calcineurin, a calcium- and calmodulin-dependent phosphatase, in a calcium-dependent manner. Inhibition of calcineurin activity is implicated in the activation and/or translocation of NFAT which binds to an IL-2 enhancer allowing the interleukin-2 gene to be transcribed. The transcription of several other cytokines, including interferon-gamma, and several other interleukins, is also inhibited by CsA.

CsA is toxic at levels necessary to mediate posttransplant graft acceptance. The primary adverse effect is renal toxicity. For example, increases in renal vascular resistance, decreased Na+ urinary excretion and other indicators of renal stress were significant after dosages of 4 and 5 mg/kg in human subjects (Butterly et al., 1995, Kidney International 48:337-343). Specific suppression of T lymphocyte activation was only observed following at or above 5 mg/kg (De Nicola et al., 1995, Nephrology, Dialysis, Transplantation 10:1739-44). In order to achieve immunosuppression, generally toxic doses of CsA must be used. In constrast, a combination of EGb761 with FK506 was found to decrease the dosage of FK506 required to achieve a comprehensive cardioprotective effect by a factor of 3-5 fold less than the unsupplemented required dosage. A immunosuppressive treatment program in which an immunophilin-binding immunosuppressive agent such as CsA or FK506 is coadministered with a gingkolide allows post transplant immunosuppression to be achieved at dosages of immunophilin-binding durg which is 3-5 times less than are currently used (i.e., in the absence of the gingkolide). In addition to augmenting immunosupppression by inhibition of T lymphocyte activity, the combination of ginkgolides plus calcineurin inhibitors counteracts vasoconstriction and postoperative reperfusion injury, which are known side effects of immunophilin-binding immunosuppressive agents. The combination therapy approach is useful to augment current therapeutic approaches to post-operative reperfusion injury of major organ systems.

Cyclosporine A and related compounds include (gamma-OH) MeLeu(4)-Cs (211-810), D-Sar (alpha-SMe)(3) Val(2)-DH—Cs (209-825), Cyclosporin G (Norvaline is substituted for alpha-aminobutyric acid at the 2 position), A238L ( a viral protein from African swine fever virus acts like cyclosporin A), SDZ 211-811, Cain ( a 240-kDa endogenous cytoplasmic protein), PD 144795 (a calcineurin-inhibitor with anti-HIV properties), sodium orthovanadate, SDZ ASM 981, cyclolinopeptide A (CLA; cyclic hydrophobic nonapeptide present in linseed), A457-482 (an autoinhibitory peptide of calcineurin), and Cypermethrin.

### FK506 and Analogs

The macrocyclic immunosuppressant FK506 (tacrolimus) is calcineurin inhibitor that has been used clinically in organ transplant surgery and for the treatment of psoriasis and rheumatoid arthritis. FK506 enters the cytoplasm of cells and forms a complex with the immunophilin FK-binding protein-12 (FKBP-12). The protein-drug complex prevents calcineurin, a calcium and calmodulin-dependent phosphatase, from producing NFAT, thereby inhibiting L-2 production.

### Rapamycin and Analogs

Rapamycin is a macrocyclic compound related to the immunosuppressant FK506. Rapamycin, like FK506, binds to FKBP-12 in the cytosol of T-cells, however, rapamycin inhibits the proliferation of T-cells through a different mechanism and at a different point in the signal transduction pathway of T-cells. The rapamycin-FKBP-12 complex interfers with the signal transduction pathway at the point by blocking IL-2 induced activation of p70 S6 kinase. The complex then prevents the phosphorylation of ribosomal S6, thereby preventing T-cell proliferation.

The formation of a complex of a drug (e.g., CsA, FK506, rapamycin, and derivatives thereof) and an immunophilin (e.g, and FKBP) is required for immunosuppressive activity. The drug can not act alone, nor can the immunophilin. For example, formation of a CsA/cyclophilin complex causes CsA to undergo a conformational change leading to exposure of hydrophobic regions of CsA. The hydrophobic regions bind to and inhibit calcineurin. FK506 and rapamycin are similar to CsA in this respect. Each of these drugs contain two distinct protein binding regions that enable them to bind both to their corresponding immunophilin and the specific signal protein on which they act.

### Gingkolides

Ginkgo Biloba L. is a plant, the leaves, roots, and fruit of which have been used for medicinal purposes for centuries. Extracts of various parts of the plant are commercially available. A gingkolide is a Ginkgo biloba extract, synthetic derivative thereof, or purified component thereof. A gingkolide preferably contains one or more immunologically active components such as an antioxidant component, a PAFR antagonist component, or a phosphodiesterase inhibitory component. For example, an extract is made from gingko leaves and used at a concentration that contains about 24-25% ginkgo-flavone-glycosides. The extract may also contain terpenoids such as Egb761 or LI-1370. For example, the preparation contains 24% ginkgo-flavone glycosides and 6% terpenoids. The ginkgo-flavone glycosides are sometimes referred to as heterosides. EGb761 is a commercially available leaf extract of Ginkgo biloba, containing: GA, GB, GC, GJ, GM and bilobalide.

A ginkgolide compound is purified when it is removed from the substances with which it naturally occurs. For example, a purified ginkgolide compound is at least 85% of the composition by weight. Preferably, the component is at least 90%, 95%, 99%, or 100% of the composition.

Naturally-occurring Ginkgo biloba contains: (A) biflavones such as amentoflavone, bilobetin, sequoiaflavone, ginkgetin, isoginkgetin, Sciadopitysin; (B) flavonol glycosides; (C) terpene trilactones, such as Gingkolide A, GingkolideB, GingkolideC, GingkolikleJ, GingkolideM and bilobalide; (D) rutin; (E) quercetin; and (F) a 30 kDa Ginkgo biloba glycoprotein, which reacts with antiserum against beta 1→2 xylose-containing N-glycans. Each component or combinations thereof are isolated from crude extracts of the plant using methods known in the art.

Alphabetically-labeled series of ginkgolide derivatives are further characterized as follows. Ginkgolide A (GA) is a leaf extract containsin terpene trilactone. This gingkolide is a PAFR antagonist, but has no apparent antioxidant properties. It is also known as BN52020, CAS 15291-75-5. Ginkgolide B (GB) is a leaf extract containing terpene trilactone. It is a PAFR antagonist, with antioxidant properties and may be referred to as BN52021 or CAS 15291-77-7. GC, ginkgolide C: a terpene trilactone, leaf extract. A PAFR antagonist, with antioxidant properties. Ginkgolide J (GJ) is a leaf extract containing terpene trilactone with PAFR antagonist activity and antioxidant properties. Ginkgolide M (GM) is a root extract containing terpene trilactone. This gingkolide has PAFR antagonist activity and antioxidant properties. Bilobalide (a sesquiterpene trilactone) is primarily an antioxidant. Ginkgo biloba extract (EGb 761) is a clinically safe, nontoxic, and easily-produced product with a wide range of applications. A synthetic gingkolide compound, hetrazepine derivative BN 50730 (Guinot, P., 1994, Clinical Reviews in Allergy 12, 397-417; U.S. Pat. No. 6,124,266)) shows more potent PAFR antagonistic activity (up to several 10-fold) compared to BN52021. Other extracts and preparation of gingkolides are known in the art, e.g., as described in Chen et al., 1998, Bioorganic & Medicinal Chemistry Letters 8:1291-6.

The gingkolide compositions to be administered are in a form which maximizes ginkgolide bioavailability: For example, the composition is a variation of EGb 761 containing 27% ginkgo-flavonol glycosides, 7% terpene lactones. This composition has been shown to extend bioavailability of pharmacologically active ginkgolide components (Li et al, 1997, Planta Medica. 63:563-5.

PAFR antagonist activity is measured using known methods, e.g., a biological assay used to evaluate the ability of a compound to inhibit the enzyme 5-lipoxygenase. For example, arachidonic acid is topically applied to a mouse ear. On application, arachidonic acid is converted by 5-lipoxygenase to various leukotrienes, which induce changes in blood flow, erythema, and increase vasodilation and vasopermeability. A candidate PAFR antagonist is similarly administered. The resulting edema is measured by comparing the thickness of the treated ear to a control ear (in the absence of a candidate compound). Reduction of the the edematous response in the presence of the compound indicates that the compound has PAFR antagonist activity. Other assays include those described by Gozal et al., 1998, Am. J. Physiol. 275 (2 Pt 2):R604-11.

Phosphodiesterase activity is measured and inhibitors of cAMP-specific phosphodiesterases are identified using methods known in the art, e.g., Bardelle et al., 1999, Anal. Biochem. 275:148-55. Oxygen free radical scavenging/antioxidant activity is also measured using methods known in the art, e.g., the method of Burits et al., 2001, Phytother. Res. 15:103-8.

BN52021 has both PAFR antagonist activity and antioxidant activity, but does not possess cAMP phophodiesterase activity. Egb 761 posesses cAMP phophordiesterase inhibitory activity, PAFR antagonist activity, as well as free radical scavenging (antioxidant) activity.

### Gingkolide Biflavonoid Compositions

Biflavones or biflavonoid compounds are polyphenolic compounds found in vascular plants. These compounds have analgesic properties and are useful as anti-inflammatories.

Amentoflavone has antifungal and antiviral effects (including activity against HIV). It has been shown not to internalize into the cell and remains associated with the membrane, where it contributes to membrane stability. Amentoflavone, like caffeine, causes a concentration-dependent increase in Ca2+ release from the heavy fraction of fragmented sarcoplasmic reticulum of rabbit skeletal muscle. The Ca2+-releasing activity of amentoflavone was approximately 20 times more potent than that of caffeine. It is an anti-inflammatory, found to inhibit cyclooxygenase (from guinea-pig epidermis) without affecting lipoxygenase. This biflavonoid inhibits group II phospholipase A2 (PLA-2) activity.

Bilobetin inhibits PLA-2, in turn inhibiting the production of TNFalpha, iNOS, and inducible cyclooxygenase (COX-2). Yet another gingko-derived biflavonoid is sequoiaflavone.

Ginkgetin inhibits PLA-2, in turn inhibiting the production of TNFalpha, iNOS and inducible cyclooxygenase (COX-2). This fraction inhibits pathogenesis of arthritis.

Other gingkolide biflavonoids include isoginkgetin, sciadopitysin, rutin, and quercetin Ginkgolide biflavonoids are inhibitors of cAMP-phosphodiesterase. Ginkgo biloba biflavones inhibit cAMP-specific phosphodiesterase in the following order of potency: amentoflavone>bilobetin>sequoiaflavone>ginkgetin=isoginkgetin. Sciadopitysin was almost inactive. (Saponara et al., 1998, Journal of Natural Products 61:1386-7).

Ginkgolide biflavonoids have been shown to have a cardioprotective effect. The influence of the main flavonoids from Crataegus species (hawthorn, Rosaceae) on coronary flow, heart rate and left ventricular pressure was investigated. Cardioprotective effects were observed with treatment of O-glycosides luteolin-7-glucoside (186%), hyperoside (66%) and rutin (66%). The data showed an inhibition of the 3′,5′-cyclic adenosine monophosphate phosphodiesterase and suggest an inhibition of this enzyme is a mechanism of cardioprotection of flavonoids (Schussler et al. 1995, Arzneimittel-Forschung 45:842-5).

The biflavonoid components of EGb761 have been shown to posess cAMP phosphodiesterase-inhibitory activity, wherease BN52021 does not have cAMP phosphodiesterase inhibitory activity. BN52021 does not contain the biflavonoids, bilotetin and gikgetin, which are present id EGb 761. Inhibition of PLA-2, TNFalpha, COX-2 and iNOS contribute to immune suppressive effect of biflavonoid-containing gingkolides, e.g., EGb 761. BN52021 does not contain biflavonoids

### Immunosuppressive Drug Combinations

The dose-response curve of a calcinerin inhibitor, e.g., FK506, was found to be favorably altered in the presence of a ginkgolide. Allograft rejection is therefore inhibited at a lower dose of the calcineurin inhibitor.

The combination drug therapy regimen described herein is based on the pharmacological action of compounds which when complexed to immunophilins suppress T cells with a gingkolide. Immunophilin-binding compounds include calcineurin inhibitors include FK506 (and structurally-related macrolides) and CsA (and structurally-related undecapeptides) as well as rapamycin (and: structurally-related inhibitors of TOR). Gingkolide compositions include extracts of ginkgo such as EGb761. The gingkolide alone or a combination of an immunophilin-binding immunosuppressive agent and a gingkolide are used for post-transplant immunosuppression. The combination of drugs has substantially lower toxicity than currently available drugs used for this purpose. For example, the dose of immunophilin-binding immunosuppressant required to achieve clinical immunosuppression is at least 5%, preferably at least 10%, preferably at least 25%, preferably at least 30%, more preferably at least 40%, and most preferably at least 50% less than that required for the same level of immunosuppression in the absence of a gingkolide. Immunosuppression measured using methods known in the art, e.g., by detecting inhibition of T cell activation (indicated by IL-2 production or expression of activation-related cell surface markers) or by measuring length of allograft survival. The combination drug therapy regimen is also useful as a post-operative cardioprotective agent and for long-term prophylaxis against cardiac hypertrophy.

A calcineurin inhibitor/ginkgolide combination drug offers a method for achieving immunosuppression sufficient to maintain transplanted tissue in a healthy, functional state with little or no side effects. Advantages of the invention include improved outcomes to transplant surgery (both in terms of survival as well as drug-related morbidity), decreased need for secondary hospitalization, and reduced expenditure of health care costs for transplant recipents. For example, the cardioprotective effect of a calcineurin inhibitor in a calcineurin inhibitor/ginkolide combination is a 5 fold lower that that required to achieve the same degree of cardioprotection with the calcineurin inhibitor alone. The synergistic effect in the combination therapy is applicable to immunosuppression.

### Treatment of Cardiac Hypertrophy

Every year, over half a million Americans are diagnosed with heart failure, approximately half of whom die from the condition. Although causes of heart disease are diverse, a common factor leading to the majority of cases is a progressive enlargement of the heart in response to various genetic and environmental factors. The enlargement begins as a protective response, but progresses into a state which eventually results in congestive heart failure and arrhythmias. FK506 has been used for suppression of the onset and effects of cardiac hypertrophy in animals. However, sustained use of this drug at dosages required to prevent hypertrophy would be toxic and unsuitable for routine human use. The biological pathway affected by FK506 is also responsive to gingkolides. Accordingly, the compositions and methods of the invention are useful for improved cardioprotection compared to the cardioprotective effect of immunophilin-binding compounds or calcineurin inhibitors alone.

The combination of the ginkgolide EGb 761 with FK506 was found to inhibit cardiac hypertrophy at 5-fold lower levels of FK506 than FK506 alone. The coadministration strategy minimizes cardiac hypertrophy and decreases the incidence of ischemia/reperfusion related arrhythmias

### Immunosuppressive and Cardioprotective Compositions with Reduced Toxicity

The compositions described herein are based on the combined action of calcineurin inhibitory (also know as “TOR” inhibitory compounds) and one or more components of the gingko extract, Egb761.

Clinical administration of a calcineurin inhibitory substance, plus a ginkgolide-derived PAFR antagonist, phosphodiesterase inhibitor, or antioxidant reduces dosage of the calcineurin inhibitor required to achieve a desired immunosuppressive effect in mammals following organ transplantation. Clinical administration of a calcineurin inhibitory substance plus a ginkgolide-derived PAFR antagonist, phosphodiesterase inhibitor, or antioxidant also positively affects cardiac functions in mammals via the same calcium-dependent signalling pathways which mediate immunosuppression.

A common biochemical pathway underlies the clinical conditions of graft rejection and cardiac dysfunction. A rise in calcium in either T lymphocytes or cardiac myocytes, respectively, causes calmodulin-dependent activation of the phosphatase calcineurin. Calcineurin activation results in processing of a class of transcription factor precursors, NFATn, which then translocate into the nucleus. In T lymphocytes, these factors cause gene expression resulting in immune activation and ultimately graft rejection. In cardiac tissue, NFATn factors cause cellular changes which result in cardiac dysfunction.

### Platelet Activating Factor (PAF)/Calcium/Calcineurin-Dependent Protection

Calcium availability to calcineurin is reduced by supplementing treatment of a subject with a calcineurin inhibitor with one or more subcomponents of Ginkgo biloba (e.g., EGb761). The gingkolide acts as an antagonist to the receptor for PAF, a potent bioactive phospholipid. The PAFR, when engaged by PAF, activates a signalling pathway causing a rise in intracellular calcium. Gingkolide compounds inhibit PAF-mediated increase in cytoplasmic calcium, thereby augmenting calcineurin inhibition by a calcineurin inhibitor such as FK506 or CsA. The low toxicity of ginkgolides allows administration of dosages of gingkolide which substantially reduce dosage of calcineurin inhibitor required for immunosuppression (or cardioprotection) with little or no side effects.

### Prevention of PAF/COX-2-Mediated Effects

PAF stimulates transcription of COX-2 (inducible prostaglandin synthase) which contributes to inflammatory damage. Ischemia of any tissue promotes PAF overproduction. PAF activity is blocked with the ginkgolide BN 50730.

### Antioxidant-Mediated Protection

The data described herein demonstrates that cardiac reperfusion-induced arrhythmias are dependent on presence of free radicals. Arrhythmias were reduced by administration of either antioxidant enzymes or ginkgolide (e.g., EGb761). Normal cardiac function is dependent on the ability of cell membranes to maintain discrete differentials of ionic species. Free radicals produced in reperfusion injury react with membrane components, leading to loss of ion separation integrity, leading to arrhythmias and other pathological effects. Antioxidants stabilize cardiac membranes with respect to their ability to maintain compartmentalization of critical ionic species. The antioxidant properties of Ginkgo biloba (e.g., the terpene component of EGb761) has cardioprotective effects. Antioxidants in EGb761 are also immunosuppressive by acting as scavengers of free radicals, thereby decreasing the degree of allograft-associated inflammatory damage.

### Amplification of Pharmacological Effect by Increasing Ginkgolide Bioavailability

EGb 761 is a standardized extract of dried leaves of Ginkgo biloba containing 24% ginkgo-flavonol glycosides, 6% terpene lactones (24/6) such as ginkgolides A, B, C, J and bilobalide. The PAFR antagonistic, phosphodiesterase inhibitory, and antioxidant effects of Egb761 confers clinical benefit, alone or when combined with a calcineurin inhibitor. For example, an immunosuppressive compound contains a calcineurin inhibitor with extract of Ginkgo biloba with a ratio of 27% ginkgo-flavonol glycosides, 7% terpene lactones (27/7), enriched in ginkgolide B. Preparation of the gingkolide portion of the composition is known in the art, e.g., the method of Li, et al., 1997, Planta Medica. 63(6):563-5.

### Coadministraton of a Calcineurin Inhibitor and Gingkolide Leads to Improved Post-Transplant Outcome

Calcineurin inhibitors, e.g., FK506, were found to act synergistically with EGb761 to mediate cardioprotection. Similar mechanisms mediate post-transplant immunosuppression. The immunological effects of Ginkgo biloba were evaluated. The results indicate that a gingkolide, e.g., EGb761, causes a decrease in T lymphocyte activation as evidenced by suppression of CD25 and CD54 expression in activated cultures. The gingkolide BN52021 was found to exhibit an anti-inflammatory effect. The mechanism of the anti-inflammatory effect is distinct from the mechanism of immunosuppression of cyclosporine A. The results indicate that combined administration of a immunophilin-binding immunosuppressive and ginkgolide leads to an increase in effective immunosuppression without a concomitant increase in toxicity. The coadministration strategy is useful to treat inflammatory disorders, incuding transplant rejection.

### Mechanisms of Immunosuppression

The compositions for immunosuppression and cardioprotection described herein include (1) a calcineurin inhibitor, (2) a PAFR receptor inhibitor, (3) a cell membrane stabilizer, and (4) a free oxygen radical scavenger. Calcineurin inhibition results in suppression of T cell activation or alternatively suppression of cardiac pathology development. PAFR inhibition, by ginkgolide components, results in diminished intracellular calcium availability, causing suppression of T cell activation or alternatively suppression of cardiac pathology development. Cardiac membrane stabilization by ginkgolide antioxidants, results in improved compartmentalization of ionic species and suppression of cardiac pathology development.

Ginkgolide antioxidants scavenge oxygen radicals released by inflammatory leukocytes participating in graft rejection pathology—reducing inflammatory damage.

### Therapeutic Administration

The results indicate that the combination of an immunophilin-binding immunosuppressive agent (e.g., a calcineurin inhibitor) and a gingkolide is useful to inhibit transplant rejection, treat cardiac hypertrophy, and to treat autoimmune diseases. The compositions are formulated into therapeutic compositions such as liquid solutions or suspensions, tablets, pills, powders, suppositories, polymeric microcapsules or microvesicles, liposomes, and injectable or infusible solutions. The preferred form depends upon the mode of administration and the particular indication targeted. The compositions also include pharmaceutically acceptable vehicles or carriers. Suitable vehicles are, for example, water, saline, dextrose, glycerol, ethanol, or the like, and combinations thereof. Actual methods of preparing such compositions are known to those skilled in the art (e.g., Remington's Pharmaceutical Sciences, Mack Publishing Company, Easton, Pa., 18th edition, 1990).

The compositions are administered using conventional modes of delivery including intravenous, intraperitoneal, oral or subcutaneous administration. In addition to systemic administration, the compositions are locally administered, e.g., to the site of an allografted tissue or a site of inflammation

Immunophilin-binding immunosuppressive agents are administered according to methods known in the art. Therapeutically effective doses are known. For example, treatment with FK506 to prevent graft rejection is conducted so as to maintain trough whole blood levels in the therapeutic range of 0.1 -1000 ng/ml (e.g., 5-20 ng/ml). This is accomplished by administration of FK506 in the range of about 0.1-5.0 mg FK506 per kg of body weight posttransplant, with dosage decreased to as low as 0.76 mg/kg for some categories of patients For most patients, treatment with this drug even at low doses (0.1 mg/kg or less), is effective in maintaining blood levels in the vicinity of 10 ng/ml—resulting in greater than 1 year graft survival for >70% of those treated. Nevertheless even at low dosage, FK506 produces chronic side effects including: hypertension, tremor, diabetes mellitus, diarrhoea and nephrotoxicity in a significant fraction of patients. Patients who experience severe acute GVHD, often require continuous intravenous infusion of FK506 for extended periods resulting in plasma concentrations of the drug as high as 25-35 ng/ml, with commensurately severe side effects. The invention provides the advantage of reducing the effective dosage of FK506 or CsA by coadministering a gingkolide. The combination of drugs augments CsA-mediated effects (while decreasing toxicity), and allows a reduction of FK506 dosage by a factor of 3-5 below what is currently perscribed with commensurate reductions in toxic side effects. The dosages of immunophilin-binding drug (and of gingkolide) may vary depending on the severity and course of the disease, the patient's health and response to treatment, and the judgment of the treating physician.

The gingkolide and calcineurin inhibitor (or rapamycin composition) are administered simultaneously or sequentially. For example, a calcineurin inhibitor is administered at a dosage of 0.1-12.0 mg/kg/day, more preferably about 0.5-6.0 mg/kg/day, and more preferably about 1.0-3.0 mg/kg/day. The selected dose is administered to a mammal in need of immunosuppression from 1-6 times daily, and is administered topically, orally, rectally, by injection, or continuously by infusion. Oral dosage units for human administration preferably contain from 0.1 to 500 mg of each active compound. Oral administration, which uses lower dosages of a calcineurin inhibitor is preferred. Parenteral administration, at higher dosages, may also be used.

Gingkolide compositions are administered in doses of 0.1 mg/kg/day to 1000 mg/kg/day. (e.g., 10 mg/kg/day-60 mg/kg/day). Routes of administration are comparable to those used for immunophilin-binding compounds such as calcineurin inhibitors.

The compositions are administered before or after transplantation of an allograft. Optionally, the allografted tissue is bathed in a solution of the immunosuppressive composition and/or the tissue is perfused with such a solution. The methods are useful for the treatment of recipients of allogeneic cells or solid organs, e.g. heart, lung, combined heart-lung, liver, kidney, pancreatic, skin or corneal transplants as well as those suffering from or at risk of developing acute rejection; hyperacute rejection, e.g., as associated with xenograft rejection; and chronic rejection, e.g., as associated with graft-vessel disease. The compositions are used to treat or prevent the development of graft-versus-host disease, such as that which may occur following bone marrow transplantation.

The immunosuppressive compositions are useful to treat or prevent autoimmune disease and of inflammatory conditions such as asthma, arthritis (e.g., rheumatoid arthritis, arthritis chronica progrediente and arthritis deformans) and rheumatic diseases. Specific auto-immune diseases for which the compositions of the invention may be employed include, autoimmune hematological disorders (including e.g. hemolytic anaemia, aplastic anaemia, pure red cell anaemia and idiopathic thrombocytopenia), systemic lupus erythematosus, polychondritis, sclerodoma, Wegener granulamatosis, dermatomyositis, chronic active hepatitis, myasthenia gravis, psoriasis, Steven-Johnson syndrome, idiopathic sprue, autoimmune inflammatory bowel disease (including e.g. ulcerative colitis and Crohn's disease) endocrine ophthalmopathy, Graves disease, sarcoidosis, multiple sclerosis, primary billiary cirrhosis, juvenile diabetes (diabetes mellitus type 1), uveitis (anterior and posterior), keratoconjunctivitis sicca and vernal keratoconjunctivitis, interstitial lung fibrosis, psoriatic arthritis, glomerulonephritis (with and without nephrotic syndrome, e.g. including idiopathic nephrotic syndrome or minimal change nephropathy) and juvenile dermatomyositis.

Individuals to be treated include any member of the class Mammalia, including, humans and non-human primates, such as chimpanzees and other apes and monkey species; farm animals such as cattle, sheep, pigs, goats and horses; domestic mammals such as dogs and cats; and laboratory animals including rodents such as mice, rats and guinea pigs. Preferably, the mammal is not a rodent such as a rat. The compositions and methods are suitatble for treatment of adult, newborn and fetal mammals. Treatment encompasses the prevention of and adverse clinical condition, e.g., transplant rejection), and the reduction or elimination of symptoms of a disease or adverse clinical condition. An immunosuppressive composition refers to any composition that suppresses or prevents an undesired immune response, e.g., prevent the immune system's rejection of a transplanted organ. Preferably, the composition reduces the activity of T cells.

The combination drug therapy described herein utilizes an immunophilin-binding immunosuppressive agent (e.g., CsA, FK506, or rapamycin) and a gingkolide composition, which contains PAFR antagonist activity and antioxidant activity. Preferably, the gingkolide compositions contains at least two antioxidant components of Gingko biloba, e.g., GB, GC, GJ, or GM, rather than one component such as GM alone. For example, the gingkolide composition is Egb761 contains several antioxidant components of Gingko biloba in addition to a component with PAFR antagonist activity. EGb761 contains a full range of antioxidants and PAFR antagonists produced by leaves of the plant. In protection of organs from immune damage, EGb761 provides a higher degree of membrane stabilization (due to oxygen radical absorption), resulting in improved regulation of ion flow (especially calcium) and less free radical-mediated tissue damage than would be expected if BN52021 were the only ginkgolide component.

Although one gingkolide GB (BN52021) has been shown to minimize the alteration of renal function induced by CsA, the present compositions and methods are distinguished over existing methods in that the ginkgolide composition is administered at a dose that is immunosuppressive (e.g., inhibits T cell activation).

### EXAMPLE 1

### Suppression of T cell Activity by Egb761

The immunosuppressive activity of BN52021 was compared with that of CsA. BN52021 is a component of EGb761. BN52021 has PAFR antagonist activity. The immunomodulatory effect of BN50521 was examined, and in vitro immune response parameters in human PBMC stimulated with EGb761 were compared to positive control stimulants such as PHA or PMA/I. BN52021 was incubated with the nonadherent fraction of PBMC cultures (i.e. primarily T and B lymphocytes).

Cultures of 2×10⁶ human PBMC were isolated from venous blood and stimulated for 24 hours with media, PHA 50 μg/ml, or PHA 50 μg/ml plus either 50 or 100 μg/ml EGb761 (Ginkgo biloba extract). Induction of CD25 (IL2R) and CD54 (ICAM-1) was measured in the cellular fractions of each culture by incubation with flurophore-conjugated monoclonal antibodies specific for each antigen, plus isotypic controls, followed by flow cytometric analysis for expression of each marker. Results are reported as percentage cells positive for either CD25 or CD54 and each measurement is the average of values obtained from 5 individual participants in this study±SE.

TABLE 1
Effect of EGb76I on PHA-induced expression of the
lymphocyte surface activation antigens CD25 and
CD54 by peripheral blood mononuclear cells in vitro.

| | | | | PHA |
|---|---|---|---|---|
| | | | | 50 μg/ml + |
| | | PHA | PHA 50 μg/ml + | EGb761 100 |
| Marker | Unstimulated | 50 μg/ml | EGb761 50 μg/ml | μg/ml |
| CD25 | 11.0 +/− 1.0 | 33.9 +/− 5.5 | 26.9 +/− 4.3 | 21.7 +/− 4.3@ |
| CD54 | 9.6 +/− 2.6 | 31.5 +/− 1.7 | 24.5 +/− 1.5* | 22.3 +/− 2.0** |

@p = 0.058 vs PHA stimulated
*p = 0.018 vs PHA stimulated
**p = 0.012 vs PHA stimulated

### EXAMPLE 2

### Combination Therapy for Asthma using a Gingkolide and a Calcineurin Inhibitor

The effects of ginkgolide B (BN52021), on in vitro activation responses of human peripheral blood mononuclear cells (PBMC) from asthmatic patients was measured. Standard methods, e.g., 2 channel flow cytometric analysis of activation-associated cell surface antigens, or ELISA assays, were used to detect cytokines known to be expressed by PBMC during T₁ or T₂ immunological activation. BN52021 is an anti-inflammatory extract of Ginkgo biloba and has been used therapeutically. BN52021 inhibits PAF, which is important in the pathogenesis of asthma. The data described herein indicate that gingkolides synergize with CsA to inhibit pathogenic immune activation in asthmatics.

The inhibitory effects of BN52021 and CsA (1 μM/L each) on activation of PBMC of asthmatic patients were measured. The cells were stimulated by phorbol myristate acetate (PMA) and calcium ionophore (IOM) and contacted with the gingkolide and/or CsA. Inhibition of production of the cytokines IL-4 and IL-5 by BN52021 was insignificant compared to CsA. However, BN52021 significantly reversed the increase in activation-associated CD45RA expression, with a trend towards decreased expression of HLA-DR. Lymphocyte activation markers were not significantly altered by CsA. The mechanism of immunosuppression of gingkolides is different from that of calcineurin inhibitors, and coadministration of a gingkolide and a calcineurin inhibitor results in at least an additive immunosuppressive effect. In some cases, the effect is synergistic.

### Patients

Subjects for the study included 9 Kuwaiti patients diagnosed with atopic asthma, 4 male and 5 female, ranging in age from 19 to 32 (mean 26±1.72). Disease duration ranged from 2 to 15 years. Atopy was defined on the basis of one or more positive skin prick tests to a range of 20 allergens. None of the patients had received systemic therapy for at least 6 weeks prior to blood collection. The mean serum IgE was 350 (172-520) IU/ml.

### Cell Cultures

Venous blood was collected in polyethylene tubes containing EDTA between 9 and 10 a.m. PBMC were separated by Ficoll-paque (Pharmacia, Uppsala, Sweden) density gradient centrifugation. Cells were washed and suspended in AIM-V medium (Gibco BRL, Gaithersburg, Md.) at density of 2×10⁶ cells/ml. PBMC were stimulated with 20 nM phorbol 12-myristate 13-acetate (PMA) and 1 μM ionomycin (IOM) (ICN Pharmaceuticals, Inc. CA, USA). The cells were harvested 24 hours later and supernatants were frozen at −70° C. until use. CsA (Sigma, St Louis, Mo.) or BN52021 (ICN Pharmaceuticals, Inc. Calif., USA) (1 μM each) were added at the start of culture; this concentration resulted in complete suppression of IL-5 expression by CsA. For BN52021, a range between 1×10⁻⁹ and 1×10⁻⁴ M was used. No change occurred in lymphocyte activation markers with 1×10⁻⁵ and 1×10⁻⁷ M in 3 of the patients tested. Based on this data, a dose of 1×10⁶ M, which dose produced significant changes, was used in subsequent experiments.

### Cytokine Expression Assays

Cytokine expression (IL-4 and IL5) was evaluated in culture supernatants by enzyme-linked immunoabsorption assay (Amersham North America, Illinois, USA). The detection range was 10-400 pg/ml and 7.8-500 pg/ml respectively. Colorimetric changes proportional to supernatant cytokine concentration were measured using a model 2550 EIA reader (Bio-Rad Laboratories, Hercules Calif. USA). All assays were performed in duplicate.

### Flow Cytometric Analysis

Two-color flow cytometry was conducted using a Coulter Epics Profile II flow cytometer (Coulter Scientific, Hialeah, Fla., USA). Isotypic controls for the monoclonal antibodies (mAb) used to detect antigens of interest were established for each cell preparation. Positive analysis regions for cells expressing specific surface antigens were set against isotypic controls and specific binding of fluorophore-conjugated mAb was analyzed by cytofluorography according to standard methods. Lymphocyte subpopulations were identified by position on forward and side scatter plots and live-gated. Isotype matched antibodies were utilized to control for nonspecific fluorescence. Peripheral blood mononuclear cells from asthmatic subjects were labeled with fluorophore-conjugated, non-specific antibody, chain-matched with the antibody to be used for a particular assay. Background values for antibodies used in assays were based on fluorescence of non-specific antibodies. Monoclonal antibodies specific for human CD25, CD45RA, CD54, HLA-DR were purchased from Dakopatts, A/S, Glostrup, Denmark. Expression of each antigen is reported as percentage cells positive for a particular surface marker plus or minus standard error.

### Statistical Analysis

Statistical analysis was performed using an independent t-test. All statistical analysis were performed using the SPSS for Windows statistical package (Norusis/SPSS, Inc.). A value of p<0.05 was considered statistically significant.

### Effects of CsA and BN52021 on Cytokine Expression

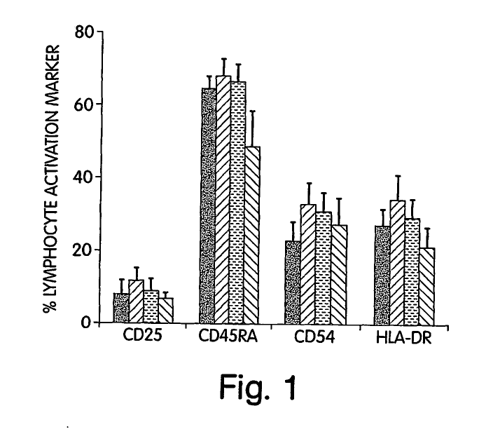

FIG. 1 and Table 2 show the cytokine levels in culture supernatants produced by cells under each stimulation condition. Treatment of PBMC with PMA and IOM induced significant increases in cytokine expression by cells extracted from all subjects, resulting in approximately a three-fold increase in expression of IL-4 (p=0.013) and an approximate four-fold IL-5 increase (p=0.0005) when results were averaged. Presence of CsA in PMA/IOM-stimulated cultures was observed to suppress expression of both cytokines (p0.008 and p=0.0005 for IL-4 and IL-5 respectively). In contrast, BN52021 had no statistically significant effect on expression of either cytokine.

The data shown Table 2 was obtained using cultures of 2×10⁶ human PBMC, which were isolated from venous blood and stimulated 24 hours with media, 20 nM PMA+1.0 μM ionomycin (PMA/I), 1.0 μM cyclosporine A+PMA/IOM, or 1 μM ginkgolide BN52021+PMA. Expression of interleukins 4 and 5 were measured in PBMC culture supernatents by antibody capture ELISA. Induction of surface antigens of interest was measured in the cellular fractions of each culture by incubation with flurophore-conjugated monoclonal antibodies specific for CD25, CD45RA, CD54 and HLA-DR, plus isotypic controls, followed by flow cytometric analysis for expression of each marker. Results of cytokine analyses are given as pg/ml±standard error (SE); and expression of lymphocyte surface markers was reported as percentage cells positive for a particular surface marker±SE.

TABLE 2
IL-4 and IL-5 expression by peripheral blood mononuclear
cells and lymphocyte surface antigen expression
in asthma patients

| | | | | PMA + IOM + |
|---|---|---|---|---|
| | Unstimulated | PMA + IOM | PMA + IOM + CyA | BN52021 |
| IL-4 (pg/ml) | 34.4 ± 4.5 | 87.9 ± 21.3¥ | 30.0 ± 4.0** | 81.7 ± 28.1 |
| IL-5 (pg/ml) | 92.5 ± 13.2 | 440.5 ± 72.2¥¥¥ | 96.2 ± 12.9*** | 374 ± 105 |
| % CD25 | 8.07 ± 3.5 | 11.8 ± 3.5 | 9.0 ± 2.7 | 7.1 ± 2.0 |
| % CD45RA | 64.8 ± 3.8 | 68.6 ± 4.1 | 66.9 ± 4.2 | 48.8 ± 9.5* |
| % CD54 | 23.3 ± 4.7 | 33.1 ± 5.3 | 31.1 ± 5.3 | 27.4 ± 6.9 |
| % HLA-DR | 27.2 ± 3.9 | 34.5 ± 6.5 | 29.5 ± 5.2 | 21.2 ± 5.3¶ |

¥P = 0.0 13 versus unstimulated cells
¥¥¥p = 0.0005 versus unstimulated cells
*p = 0.042 versus PMA/IOM-treated cultures
**p = 0.008 versus PMA/IOM-treated cultures
***p = 0.0005 versus PMA/IOM-treated cultures
¶p = 0.067 versus PMA/IOM-treated cultures

### Effects of Cyclosporin A and BN52021 on Expression of Lymphocyte Activation Markers

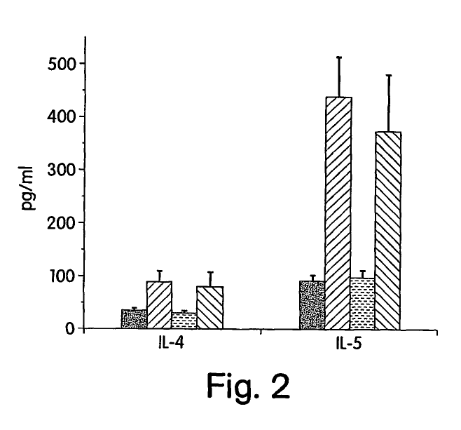

As shown in FIG. 2 and Table 2, stimulation of cultures with with PMA/IOM resulted in an increase in expression of CD25, CD54 and HLA-DR versus unstimulated controls, while CD45 was unaffected. Nevertheless, individual responses to this reagent varied: cells from 4 of the 9 subjects responded by upregulation of CD25 and HLA-DR; cells from 3 subjects showed expanded fractions of either CD25 or HLA-DR, but not both when treated with PMA/IOM; and cells from 2 subjects failed to upregulate either CD25 or HLA-DR in response to PMA/IOM. However, cellular activation was confirmed by the substantial increase in large, blast cells for all PMA/IOM-stimulated cultures. These results indicate that PMA/IOM is a valid positive control stimulant for each of the variables. CsA caused no changes in cell surface activation antigen expression. CD25 and CD54 expression in cells treated with BN52021 was unaffected, but expression of CD45RA was decreased significantly (p=0.042) with BN52021, and a trend towards the reduction of HLA-DR expression (p=0.067) was seen.

Compared to CsA, BN52021 showed different effects on cytokine production and surface antigen expression by PBMC from asthma patients stimulated with PMA and inonomycin. CsA completely inhibited IL-4 and IL-5 production whereas BN52021 had very little effect. BN52021, but not CsA, significantly reduced CD45RA and showed a trend towards reduced expression of HLADR. CD25, CD54 were not significantly changed with either agent. The profound effect of CsA on IL-4 and IL-5 expression is mediated by its direct inhibition of calcineurin. BN52021 fails to effectively suppress calmodulin/calcineurin dependent expression of IL-4 and IL-5. In contrast it suppressed expression of CD45RA and also appeared to reduce the frequency of HLA-DR+ cells, in both cases, to below the values for unstimulated cells. This effect was not seen with CsA. PAFR antagonism of the ginkgolide fails to reduce intracellular calcium to levels at which calcineurin would be substantially inhibited allowing continued production of IL-4 and IL-5. The pathway by which this compound exerts its effect on cellular activation differs from the CsA-sensitive path, especially as the latter induced widely contrasting effects on cell surface antigen expression.

The potent PAF receptor antagonist, BN52021 has been shown to have beneficial clinical effects on aspects of asthma pathogenesis mediated by PAF. The mechanism by which it modulates surface antigen expression during lymphocyte activation is likely due to selective elimination of CD45RA⁺ cells or a shift in the equilibrium towards CD45RO⁺ lymphocytes. Certain states of lymphocyte activation may be associated with enhanced dual expression of CD45RO and RA, and the reduction of RA expression represents a suppressive immunomodulatory effect. Circulating CD45RA⁺/CD45RO⁺/HLADR+ cells which are increased in atopic asthma area transitional cellular phenotype, leading ultimately to a pathogenic T cell. The data indicate that, by its inhibition of CD45RA and HLA-DR, BN52021 exerts a clinical benefit by suppression of the development of these cells.

Very little toxicity is associated with ginkgolides, and no adverse immune effects have been reported in response to these compounds. This class of immunosuppressive agents are useful, alone or in combination with other immunosuppressive agents, for treatment of inflammatory disorders such as asthma, in which substantial tissue damage occurs by immunological processes involving PAF expression and free radical production.

BN52021 modulate lymphocyte activation in asthmatics. When grouped responses of each stimulation condition are considered, the data demonstrate that the ginkgolide BN52021 inhibits immunological activity in leukocytes from asthma patients by a mode different from that of cyclosporine. These data indicate that this class of drug is useful as a therapeutic agents to inhibit T cell activity or to augment the inhibition of T cell activity mediated by CsA and other calcineurin inhibitors.

### EXAMPLE 3

### Cardioprotective Effect of a Calcineurin Inhibitor and a Gingkolide

Malignant ventricular arrhythmias, often resulting in sudden cardiac death, have been observed to arise as a result of postischemic reperfusion injury secondary to heart diseases, particularly in cases characterized-by persistent angina pectoris and also as a consequence of some clinical interventions, including angioplasty, saphenous vein bypass grafting, release of coronary spasm and thrombolytic therapy. Free radicals released into the myocardium during postischemic reperfusion are significant contributors to development of arrhythmias and impairment of cardiac function. Insufficient cardiac function and arrhythmias may also arise as a result of myocardial hypertrophy developing as a consequence of hypertension, endocrine disorders and genetic mutations in cardiac contractile elements. Each factor contributing to the development of a hypertrophic response by the cardiomyocytes, is observed to cause an increase in intracellular calcium levels. The rise results in calmodulin-dependent activation of calcineurin, which acts on the cytoplasmic precursor of the transcription factor NFAT₃, to allow its translocation into the nucleus, where it interacts with the cardiac-restricted, zinc finger transcription factor GATA4, eventually giving rise to cardiac hypertrophy.

Administration of an immunophilin-binding immunosuppressive agent and a gingkolide results in a synergistic cardioprotective effect. Effects of the calcineurin inhibitor FK506, the PAF antagonist and free radical scavenger Ginkgo biloba extract, EGb 761, and their combination on reperfusion-induced ventricular fibrillation (VF), ventricular tachycardia (VT) and recovery of cardiac function were studied after 30 minutes of global ischemia followed by 2 hours of reperfusion in isolated rat hearts. In the first series of studies, rats received a daily (oral) dose of 0, 1, 5, 10, 20 or 40 mg/kg/day FK506 for 10 days. FK506 dose-dependently reduced the incidence of reperfusion-induced total (irreversible plus reversible) VF from a value of 92% for untreated animals, to: 92% (NS), 83% (NS), 67% (NS), 33% (p<0.05), and 25% (p<0.05), for doses of 1-40 mg/kg/day respectively, with effects on incidence of VT showing the same pattern. FK506 between 20 and 40 mg/kg/day also resulted in significant recovery of postischemic cardiac function. In the second series of studies, rats were treated with or EGb761 alone or with the combination of FK506. Whereas no significant reduction in arrhythmias or improvement in cardiac function resulted from single intervention of EGb761 at 25 mg/kg/day, combined treatment of rats with 25 mg/kg/day of EGb761 and 1 mg/kg/day or 5 mg/kg/day of FK506 resulted in a reduction in total and irreversible VF of 92% and 92% to 42% (p<0.05) and 33% (p<0.05), 25% (p<0.05) and 8% (p<0.05), respectively, versus untreated control animals; paralleled by similar effects seen on the incidence of VT and accompanied by significant improvements in postischemic cardiac function. The results demonstrate a cardioprotective characteristic of FK506 and indicate that combination therapy using FK506 plus EGb 761 synergistically improves postischemic cardiac function, while reducing incidence of reperfusion-induced VF and VT. The synergistic activity of FK506 and a gingkolide allows therapy with FK506 for cardioprotection as well as immunosuppression for transplant recipients and individuals suffering from autoimmune disease, at lower doses of FK506 than are currently used.

### Animals

Male Sprague-Dawley rats were used for all studies.

### Isolated Working Heart Preparation

Rats were anesthetized with intraperitoneal pentobarbital sodium (60 mg/kg body weight) and then given intravenous heparin (500 IU/kg). After thoracotomy, the heart was excised and placed in ice cold perfusion buffer. Immediately after preparation, the aorta was cannulated, and the heart was perfused according to Langendorf method for a 5-min washout period at a constant perfusion pressure equivalent to 100 cm of water (10 kPa). The perfusion medium consisted of a modified Krebs-Henseleit bicarbonate buffer: 118 mM NaCl, 4.7 mM KCl, 1.7 mM CaCl₂, 25 mM NaHCO₃, 0.36 mM KH₂PO₄, 1.2 mM MgSO₄, and 10 mM glucose. The left atrium was cannulated and the Langendorff system was switched to the working mode with a left atrial filling pressure of 17 cm of buffer (1.7 kPa) and aortic afterload pressure of 100 cm (10 kPa) of buffer. Aortic flow was measured by a calibrated rotameter, and coronary flow rate was measured by a timed collection of the coronary perfusate that dripped from the heart.

### Induction of Ischemia and Reperfusion

After 10 min aerobic perfusion of the heart, the atrial inflow and aortic outflow lines were clamped at a point close to the origin of the aortic cannula. Reperfusion was initiated by unclamping the atrial inflow and aortic outflow lines. To prevent the drying out of the myocardium during global ischemia, the thermostated glassware (in which hearts were suspended) was covered and the humidity was kept at a constant level (90%-95%).

### Indices Measured

An epicardial ECG was recorded by a polygraph throughout the experimental period by two silver electrodes attached directly to the heart. ECGs were analyzed to determine the incidence of ventricular fibrillation (VF) and ventricular tachycardia (VT) and whether VF was non-sustained (spontaneously reverting to regular rhythm) or sustained (persisting through the first 3 min of reperfusion). After 3 min of ventricular fibrillation (sustained VF) hearts were defibrillated and myocardial function was recorded. The heart was considered to be in VF if an irregular undulating baseline was apparent on the ECG. VT was defined as 5 or more consecutive premature ventricular complexes; and this classification included repetitive monomorphic VT, which is difficult to dissociate from rapid VT. In each instance, VT spontaneously switched to sinus rhythm or VF, therefore VT was considered non-sustained. The heart was considered to be in sinus rhythm if normal sinus complexes occurring in a regular rhythm were apparent on the ECG. Before ischemia and during reperfusion, heart rate (HR), coronary flow (CF) and aortic flow (AF) rates were registered. Left ventricular developed pressure (LVDP) which was defined as the difference between left ventricular systolic and end-diastolic pressure, and the first derivative of LVDP (+LVdp/dtₘₐₓ) were also recorded (Experimetria, UK) by the insertion of a catheter into the left ventricle via the left atrium and mitral valve.

### Experimental Time Course

In the first series of the study, rats (n=12 in each group) were orally treated with various doses of FK506 (0, 1, 5, 10, 20, and 40 mg/kg/day) for 10 days. After 24 hours of the last treatment, hearts were excised, isolated, and subjected to 30 min of global ischemia followed by 2 hours of reperfusion. In the second series of the study, rats were orally treated with 25 mg/kg/day of EGb 761, 25 mg/kg/day of EGb 761 plus 1 mg/kg/day of FK506, 25 mg/kg/day of EGb 761 plus 5 mg/kg/day of FK506 for 10 days, respectively. Twenty four hours following the last treatment, hearts were isolated and the ischemia/reperfusion protocol (30 min. ischemia and 2 hours reperfusion) was conducted. The incidence of arrhythmias (VF and VT) and cardiac function (HR, CF, AF, LVDP, and +dP/dtₘₐₓ) were registered.

### Exclusion Criteria

Preselected exclusion criteria for the present studies demanded that hearts were excluded if: (I) ventricular arrhythmias occurred during the period prior to the induction of global ischemia, (II) coronary flow and aortic flow rates were less than 17 ml/min and 40 ml/min, respectively, prior to the induction of ischemia. These criteria led to exclusion and replacement of 9 hearts in the study.

### Statistics

Cardiac function data (HR, CF, AF, LVDP, and LVdp/dtₘₐₓ), were expressed as the mean±SEM. One-way analysis of variance was first carried out, to test for any differences between the mean values of all groups. If differences were established, the values of the control, drug-free group were compared with those of the drug treated groups by a two-tailed t-test with the Bonferroni correction (Wallenstein et al., 1980, Circ. Res. 47:1-9). An analogue procedure was followed for distribution of discrete variables such as the incidence of VF and VT. An overall chi-square test for a 2×n table was constructed followed by a sequence of 2×2 chi-square tests to compare individual groups. A change of p<0.05 was considered significant.

### Effects of FK 506 on Arrhythmias and Cardiac Function

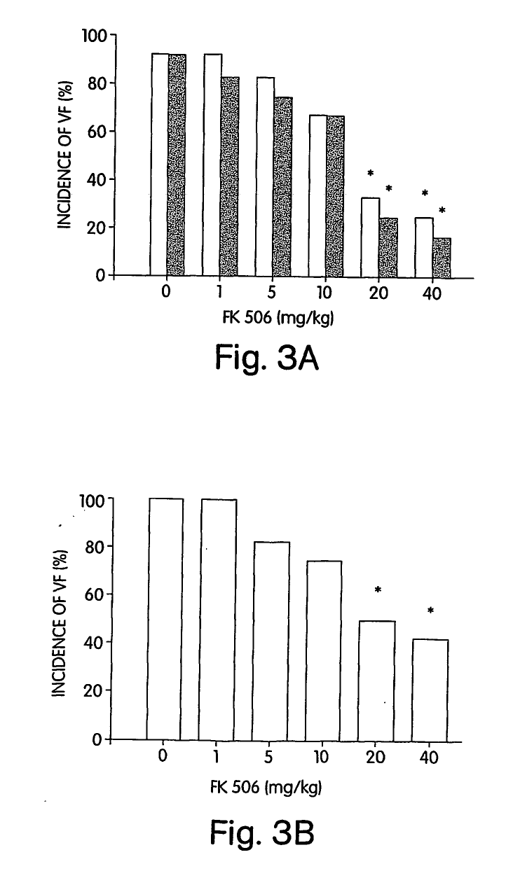

The control group was selected to exhibit a high vulnerability to reperfusion-induced arrhythmias, in order that maximum scope would exist for the demonstration of antiarrhythmic effects of FK 506 and its combination with EGb 761. To ensure this within the experimental time course and conditions defined for this study, 30 min normothermic global ischemia followed by 2 hours reperfusion was selected. The results demonstrate (FIGS. 3A-B) that in rats treated with different doses of FK 506, the incidence of reperfusion-induced arrhythmias was dose-dependently reduced. Thus, with 1, 5, 10, 20, and 40 mg/kg of FK 506, the incidence of total (irreversible plus reversible) VF was reduced (FIG. 3A) from its drug-free control value of 92% to 92% (NS), 83% (NS), 67% (NS), 33% (p<0.05), and 25% (p<0.05), respectively. The incidence of irreversible VF (FIG. 3A, solid columns) and the incidence of VT (FIG. 3B) showed the same pattern. Animals showed excellent tolerance of FK506, with no visible evidence of toxicity observed, even at the high end of the dosage range used.

In the drug-free group, the preischemic values of HR, CF, AF, LVDP and +LVdP/dtₘₐₓ were 305±8 beats/min., 27.3±0.8 ml/min., 49.9±1.4 ml/min., 18.2±0.3 kPa 807±21 kPa/s respectively. No significant changes were observed in these values as a result of FK506 treatment. Table 3 shows the absolute values for postischemic cardiac function in the drug-free control and FK 506 treated groups. Thus, a significant recovery of postischemic cardiac function (CF, AF, LVDP, and LVdP/dtₘₐₓ), was observed in the groups treated with 20 and 40 mg/kg of FK 506, respectively. Heart rate did not show a significant change in the treated groups in comparison with the drug-free control values, either before the induction of ischemia or during reperfusion.

TABLE 3
The effect of FK 5406 on cardiac function after 30-min ischemia followed by reperfusion

| FK 506 | After 30-min reperfusion | | | | | After 60-min reperfusion | |
|---|---|---|---|---|---|---|---|
| (mg/kg) | HR | CF | AF | LVDP | +dP/dtₘₐₓ | HR | CF |
| 0 | 306 ± 6 | 16.8 ± 0.8 | 9.8 ± 0.8 | 10.7 ± 0.5 | 417 ± 16 | 293 ± 7 | 18.0 ± 0.7 |
| 1 | 315 ± 9 | 18.2 ± 1.0 | 10.6 ± 0.4 | 10.7 ± 0.6 | 422 ± 28 | 306 ± 8 | 16.9 ± 0.9 |
| 5 | 298 ± 7 | 18.0 ± 1.1 | 12.0 ± 0.9 | 11.0 ± 0.4 | 430 ± 34 | 282 ± 7 | 18.4 ± 0.7 |
| 10 | 308 ± 8 | 20.0 ± 1.4 | 12.0 ± 1.2 | 11.5 ± 0.5 | 478 ± 44^{a} | 310 ± 9 | 19.6 ± 1.2 |
| 20 | 298 ± 9 | 23.1 ± 1.1^{a} | 17.4 ± 0.8^{a} | 13.5 ± 0.6^{a} | 536 ± 28^{a} | 297 ± 8 | 22.0 ± 1.1^{a} |
| 40 | 291 ± 7 | 24.7 ± 1.5^{a} | 20.6 ± 1.3^{a} | 14.1 ± 0.6^{a} | 557 ± 31^{a} | 286 ± 9 | 23.5 ± 0.8^{a} |

| After 60-min reperfusion | | | After 120-min reperfusion | | | | |
|---|---|---|---|---|---|---|---|
| AF | LVDP | +dP/dtₘₐₓ | HR | CF | AF | LVDP | +dP/dtₘₐₓ |
| 11.8 ± 0.6 | 11.2 ± 0.5 | 439 ± 18 | 287 ± 6 | 17.3 ± 0.5 | 10.4 ± 0.5 | 10.8 ± 0.4 | 428 ± 21 |
| 10.9 ± 0.6 | 11.9 ± 0.5 | 447 ± 26 | 302 ± 9 | 17.5 ± 0.7 | 10.1 ± 0.6 | 11.2 ± 0.3 | 432 ± 17 |
| 13.3 ± 1.0 | 12.0 ± 0.7 | 463 ± 23 | 279 ± 8 | 18.1 ± 0.8 | 11.2 ± 0.8 | 11.5 ± 0.4 | 451 ± 32 |
| 13.3 ± 1.2 | 12.3 ± 0.7 | 472 ± 32 | 307 ± 7 | 18.8 ± 0.7 | 11.4 ± 0.7 | 11.7 ± 0.6 | 463 ± 29 |
| 19.8 ± 1.5^{a} | 14.1 ± 0.6^{a} | 544 ± 31^{a} | 295 ± 6 | 21.2 ± 1.1^{a} | 17.3 ± 1.1^{a} | 13.2 ± 0.5^{a} | 519 ± 26^{a} |
| 22.9 ± 2.1^{a} | 15.3 ± 0.6^{a} | 594 ± 26^{a} | 289 ± 8 | 22.8 ± 1.8^{a} | 20.0 ± 1.8^{a} | 14.6 ± 0.6^{a} | 568 ± 33^{a} |

### Combined Treatment with FK 506 with EGb 761: Effects on Arrhythmias and Postischemic Cardiac Function

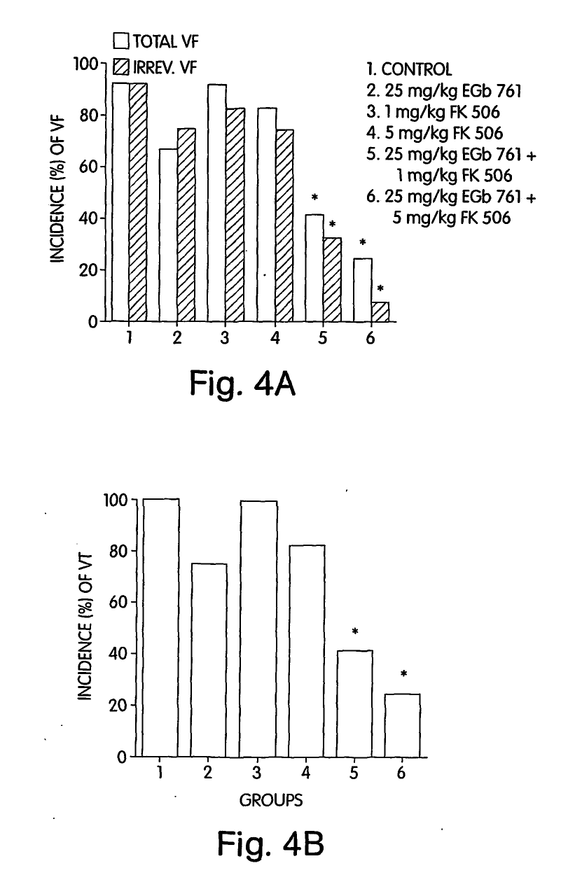

FIGS. 4A-B show that neither 25 mg/kg of EGb 761 nor 1 and 5 mg/kg of FK 506 significantly reduced the incidence (%) of reperfusion-induced arrhythmias. The combination of 25 mg/kg of EGb 761 with 1 or 5 mg/kg of FK 506 resulted in a significant reduction in the incidence of reperfusion-induced VF (FIG. 4A) and VT (FIG. 4B). Thus, the coadministration of 25 mg/kg EGb 761 with 1 and 5 mg/kg of FK 506 (FIG. 4A), significantly reduced the incidence of total VF and irreversible VF from their control values of 92% and 92% to 42% (p<0.05) and 33% (p<0.05), 25% (p<0.05) and 8% (p<0.05), respectively. This reduction followed the same pattern in the incidence of reperfusion-induced VT (FIG. 4B).

Table 4 shows that cardiac function was not significantly changed in the groups treated with a single interventions during reperfusion. The combination of 25 mg/kg EGb 761 with 1 and 5 mg/kg of FK 506, significantly improved the recovery of cardiac function (CF, AF, LVDP, and +dP/dtₘₐₓ) during reperfusion (Table 4). Heart rate was not significantly changed in the combination group either before the introduction of ischemia or during reperfusion (Table 4).

TABLE 4
The effect of FK 506 with the combination of Egb761 on cardiac function after ischemia followed

| | | | | | | After | |
|---|---|---|---|---|---|---|---|
| | After 30-min reperfusion | | | | | 60-min reperfusion | |
| Groups | HR | CF | AF | LVDP | +dP/dtₘₐₓ | HR | CF |
| Untreated | 306 ± 6 | 16.8 ± 0.8 | 9.8 ± 0.8 | 10.7 ± 0.5 | 417 ± 16 | 293 ± 7 | 18.0 ± 0.7 |
| 25 mg/kg | 294 ± 8 | 17.4 ± 0.5 | 11.0 ± 0.7 | 11.2 ± 0.5 | 425 ± 17 | 287 ± 9 | 19.1 ± 0.9 |
| Egb761 | | | | | | | |
| 1 mg/kg FK506 | 315 ± 9 | 18.2 ± 1.0 | 10.6 ± 0.4 | 10.7 ± 0.6 | 422 ± 28 | 306 ± 8 | 16.9 ± 0.9 |
| 5 mg/kg FK506 | 298 ± 7 | 18.0 ± 1.1 | 12.0 ± 0.9 | 11.0 ± 0.4 | 430 ± 34 | 282 ± 7 | 18.4 ± 0.7 |
| 25 mg/kg | 300 ± 7 | 21.5 ± 0.9^{a} | 23.9 ± 1.1^{a} | 12.9 ± 0.4^{a} | 522 ± 23^{a} | 296 ± 6 | 19.5 ± 1.2^{a} |
| Egb761 + | | | | | | | |
| 1 mg/kg FK506 | | | | | | | |
| 25 mg/kg | 309 ± 6 | 23.9 ± 1.2^{a} | 25.2 ± 1.2^{a} | 13.6 ± 0.5^{a} | 528 ± 27^{a} | 302 ± 9 | 23.2 ± 1.1^{a} |
| Egb761 + | | | | | | | |
| 5 mg/kg FK506 | | | | | | | |

| After 60-min reperfusion | | | After 120-min reperfusion | | | | |
|---|---|---|---|---|---|---|---|
| AF | LVDP | +dP/dtₘₐₓ | HR | CF | AF | LVDP | +dP/dtₘₐₓ |
| 11.8 ± 0.6 | 11.2 ± 0.5 | 439 ± 18 | 287 ± 6 | 17.3 ± 0.5 | 10.4 ± 0.5 | 10.8 ± 0.4 | 428 ± 21 |
| 12.3 ± 0.9 | 12.2 ± 0.5 | 458 ± 20 | 292 ± 8 | 18.1 ± 0.7 | 11.1 ± 0.6 | 11.5 ± 0.5 | 433 ± 19 |
| 10.9 ± 1.8 | 11.9 ± 0.5 | 447 ± 26 | 302 ± 9 | 17.5 ± 0.7 | 10.1 ± 0.6 | 11.2 ± 0.3 | 432 ± 17 |
| 13.3 ± 1.0 | 12.0 ± 0.7 | 463 ± 23 | 279 ± 8 | 18.1 ± 0.8 | 11.2 ± 0.8 | 11.5 ± 0.4 | 451 ± 32 |
| 21.8 ± 1.9^{a} | 14.1 ± 0.6^{a} | 561 ± 24^{a} | 292 ± 7 | 21.8 ± 1.0^{a} | 19.2 ± 1.9^{a} | 13.8 ± 0.6^{a} | 540 ± 30^{a} |
| 23.9 ± 1.0^{a} | 15.3 ± 0.6^{a} | 588 ± 31^{a} | 300 ± 8 | 24.2 ± 1.5^{a} | 22.4 ± 1.0^{a} | 15.0 ± 0.7^{a} | 588 ± 23^{a} |

The results described herein support a role for FK506 as an antiarrhythmic agent and additionally demonstrate its capacity to improve cardiac function. A dose-dependent improvement in cardiac function (Table 3) and inhibition of postischemic arrhythmias (FIGS. 3A-B) resulted from pretreatment of rats with FK506. It is likely that this effect is the result of FK506-mediated blockage of events downstream of calcineurin/NFAT3/GATA4. A calcineurin-independent property of FK506 may also play a role in cardioprotection. Under physiological conditions, FK506 is observed to form a complex with FKBP12, a ubiquitously expressed 12 kDa immunophilin protein, which subsequently binds to calcineurin in the presence of FK506, inhibiting its ability to dephosphorylate cytoplasmic NFAT₃ and blocking NFAT3-dependent gene expression. FKBP12 has been demonstrated to be a regulatory structural component of the ryanodine receptor intracellular calcium release channel (RyR). Calcium channels composed exclusively of RyR (with no associated FKBP12) manifest altered calcium flux across the sarcoplasmic membrane; moreover, channels incorporating FK506-bound FKBP12 exhibit abnormal calcium gating properties. Thus, a major role for FKBP12 is to modulate the calcium flux through the RyR channel complex. This function is disrupted if FK506 is additionally incorporated, causing a reversal of the channel-stabilizing effects of FKBP12, producing effects which include increased channel sensitivity to channel-opening ligands such as caffeine, elevated calcium flow and leakage of the ion across sarcoplasmic reticulum. Although, clinical use of FK506 in humans is at dosage levels too low to bind significant amounts of FKBP, the observed effects occurred at relatively high, perhaps toxic, doses of FK506, with no effects noted on VF, VT or hemodynamic recovery below 20 mg/kg. These data indicate that FK506 may possess a mode of pharmacological action separate from calcineurin inhibition (which is known to occur efficiently at dosages in the range of 0.15 mg/kg). It is likely that the increased leak of calcium occurring in the presence of FK506 alter normal pathways to cardiac excitation and lead to arrhythmogenesis. However, based on the efficient inhibition of calcineurin phosphatase at relatively low doses, it is likely that the effects are the result of FK506 interaction with Ca⁺⁺ channel proteins such as FKBP12.

Combining FK506 with other treatments which lower its dosage requirement without diminishing its cardioprotective properties allows clinical use of FK506 in prevention and therapy of cardiac disorders. The highest dosage of the ginkgolide was 25 mg/kg, which did not result in significant decreases in either VF or VT. Likewise, FK506 dosages between 1-5 mg/kg failed to affect these parameters. Combined treatment with both drugs synergistically and dose-responsively reduced the incidence of postischemic arrhythmias and additionally resulted in significant improvements in cardiac function. The molecular mechanisms contributing to these effects include diminished levels of oxygen radical-induced damage to cardiomyocyte membranes, resulting from the demonstrated antioxidant properties of EGb 761, contributing to restoration of stable compartmentalization and transmembrane flow of Na⁺, K⁺, Ca²⁺ and Mg²⁺. Coadministration to rats of EGb 761 plus antioxidant enzymes (SOD or catalase), also produced a dose-dependent reduction in reperfusion-induced arrhythmias (albeit not as dramatic as the ginkgolide-FK506 combination), paralleled by decreases in free radical concentrations as measured by DMPO adduct formation in heart perfusate buffer. The data indicate that development of cardiac hypertrophy is prevented by administration of an immunophilin-binding immunosuppressive agent and a gingkolide. The combination allows subtoxic dosage of an immunophilin-binding composition, e.g., FK506, by coadministration of a ginkgolide.

### EXAMPLE 4

### Effect of EGb761 and Cyclosporin on PHA-Induced Expression of the lymphocyte Surface Activation Antigens CD25 by Peripheral Blood Mononuclear Cells in vitro

Combined treatment with both ginkgo extract and cyclosporin synergistically inhibits immune activation in human tissue. Primary human peripheral blood mononuclear cells (PBMC) were treated with a gingkolide and cyclosporin and immune activation measured using standard methods, e.g., flow cytometry.

Cultures of 2×10⁶ human PBMC were isolated from venous blood and cultured for 24 hours under the following conditions: (i) without stimulant; (ii) with PHA, 50 μg/ml; (iii) PHA plus 200 μg/ml EGb761 (Ginkgo biloba extract); (iv) PHA plus 400 μg/ml EGb761; and (v) PHA plus 400 μg/ml EGb761, plus 1.0 μM cyclosporin. Induction of CD25 (IL2R) was measured in the cellular fractions of each culture by incubation with fluorophore-conjugated monoclonal antibodies specific for CD25, plus isotypic controls, followed by flow cytometric analysis. Results (shown in Table 5) were expressed as percentage cells positive for CD25. Each measurement is the average of values obtained from 5 individuals±SE.

TABLE 5
Percentage of cells expressing CD25 (IL-2 receptor) following stimulation with
PHA and treatment with ginkgolide (Egb) and/or cyclosporin A (CyA)

| | | | | | PHA 50 μg/ml + |
|---|---|---|---|---|---|
| Stimulation | | | PHA 50 μg/ml | PHA 50 μg/ml + | 400 μg/ml EGb + |
| conditions | Unstimulated | PHA 50 μg/ml | 200 μg/ml Egb | 400 μg/ml EGb | 1 μM CyA |
| Percent | 28.2% ± 2.1 | 34.5% ± 5.2 | 10.6% ± 0.7 | 4.5% ± 0.23 | 2.7% ± 0.41 |
| CD25 + cells | | | | | |

The data shown in Table 5 indicate that the gingkolide preparation, Egb761, alone suppresses immune activation by human leukocytes. Cyclosporin further reduces expression of CD25. The effect of the gingkolide and cyclosporin is more than additive; the addition of cyclosporin demonstrates a synergistic immunosuppressive effect of the combination of these drugs. The results also demonstrate the advantage of using this particular approach to screen a large number of candidate preparations for immunosuppressive potential. The assay is easily performed and allows a large number of samples to be processed simultaneously.

Immunosuppressive agents are identified by an immune cell population, e.g., PBMC, or a T cell line, with candidate ginkgolide compound and measuring T cell activation. The cells are activated in culture, e.g., by culturing the cells in the presence of an immunostimulatory compound such as phytohemagglutinin (PHA). A decrease in T cell activation in the presence of the compound compared to the level in the absence of the compound indicates that the compound is an immunosuppressive agent. Immune cell activation is measured using known methods, e.g., cell proliferation, expression of cell surface markers such as CD25, or elaboration of cytokines such as IL2. A method of identifying identifying a syngergistic combination of immunosuppressive compounds is carried out similarly activating cultures of immune cells (a) in the presence an immunophilin-binding compound, (b) in the presence of a candidate ginkgolide compound; and (c) in the presence of both an immunophilin-binding compound and a candidate ginkgolide compound. Immune activation in each sample is measured as described above. A greater than additive decrease in T cell activation detected in (c) compared to that detected in (a) and (b) indicates that the ginkgolide and immunophilin-binding compound tested are synergistically immunosuppressive.

Other embodiments are within the following claims.

What is claimed is:

1. A composition comprising an immunophilin-binding compound and a gingkolide compound, wherein said ginkgolide compound is selected from the group consisting of a phosphodiesterase inhibitor, a platelet activating factor (PAFR) antagonist, and a free radical scavenger.

2. The composition of claim 1, wherein said ginkgolide comprises a phosphodiesterase inhibitor.

3. The composition of claim 1, wherein said ginkgolide comprises a PAFR antagonist.

4. The composition of claim 1, wherein said ginkgolide comprises a free radical scavenger.

5. The composition of claim 1, wherein said ginkgolide is BN52021.

6. The composition of claim 1, wherein said ginkgolide is BN50730.

7. The composition of claim 1, wherein said ginkgolide is EGb761.

8. The composition of claim 1, wherein said immunophilin-binding compound is rapamycin.

9. The composition of claim 1, wherein said immunophilin-binding compound is a calcineurin inhibitor.

10. The composition of claim 9, wherein said calcineurin inhibitor is FK506 or Cyclosporin A.

11. A method of inhibiting activation of an immune cell, comprising contacting said immune cell with the composition of claim 1.

12. A method of inhibiting rejection of an allograft in a mammal, comprising administering to said mammal an immunophilin-binding compound and a gingkolide compound, wherein said ginkgolide compound is selected from the group consisting of a phosphodiesterase inhibitor, a platelet activating factor (PAFR) antagonist, and a free radical scavenger.

13. A method of inhibiting cardiac hypertrophy in a mammal, comprising administering to said mammal an immunophilin-binding compound and a gingkolide compound, wherein said ginkgolide compound is selected from the group consisting of a phosphodiesterase inhibitor, a platelet activating factor (PAFR) antagonist, and a free radical scavenger.

14. A method of reducing a symptom of an autoimmune disease, comprising administering to said mammal an immunophilin-binding compound and a gingkolide compound, wherein said ginkgolide compound is selected from the group consisting of a phosphodiesterase inhibitor, a platelet activating factor (PAFR) antagonist, and a free radical scavenger.

15. The method of claim 14, wherein said symptom is chronic inflammation.

16. A method of reducing a symptom of asthma, comprising administering to said mammal an immunophilin-binding compound and a gingkolide compound, wherein said ginkgolide compound is selected from the group consisting of a phosphodiesterase inhibitor, a platelet activating factor (PAFR) antagonist, and a free radical scavenger.

17. A method of suppressing an immune response in a mamal, comprising administering to said mammal an effective amount for inducing a synergistic immunosuppression of an immunophilin-binding compound and a gingkolide compound, wherein said ginkgolide compound is selected from the group consisting of a phosphodiesterase inhibitor, a platelet activating factor (PAFR) antagonist, and a free radical scavenger.

17. The method of claim 12, 13, 14, 16, or 17, wherein said immunophilin-binding compound and said ginkgolide compound are administered simultaneously.

18. The method of claim 12, 13, 14, 16, or 17, wherein said immunophilin-binding compound is administered prior to said ginkgolide compound.

19. The method of claim 12, 13, 14, 16, or 17, wherein said ginkgolide compound is administered prior to said immunophilin-binding compound.

20. A method of identifying an immunosuppressive agent, comprising contacting an immune cell population with candidate ginkgolide compound and measuring T cell activation, wherein a decrease in T cell activation in the presence of said compound compared to the level in the absence of said compound indicates that said compound is an immunosuppressive agent.

21. The method of claim 20, wherein T cell activation is measured by detecting cell surface CD25 expression.

22. A method of identifying identifying a syngergistic combination of immunosuppressive compounds, comprising
(a) contacting an immune cell with an immunophilin-binding compound;
(b) contacting an immune cell with a candidate ginkgolide compound;
(c) contacting an immune cell with both an immunophilin-binding compound and a candidate ginkgolide compound; and
(d) measuring T cell activation, wherein a greater than additive decrease in T cell activation detected in (c) compared to that detected in (a) and (b) indicates that said ginkgolide and said immunophilin-binding compound are synergistically immunosuppressive.
